(19) United States
(12) Patent Application Publication
(10) Pub. No.: US 2019/0231417 A1
(43) Pub. Date: Aug. 1, 2019

(54) FLOW SHEATH, RETRACTOR TOOL, AND ELECTRODE OF A RESECTOSCOPE

(71) Applicant: COVIDIEN LP, Mansfield, MA (US)

(72) Inventors: JOE D. SARTOR, LONGMONT, CO (US); DAVID N. HEARD, BOULDER, CO (US); NICK C. ORTIZ, BOULDER, CO (US)

(21) Appl. No.: 16/249,094

(22) Filed: Jan. 16, 2019

Related U.S. Application Data
(60) Provisional application No. 62/622,231, filed on Jan. 26, 2018.

(51) Int. Cl. A61B 18/14; A61B 1/00; A61L 31/02; A61L 31/06; A61M 3/02; A61M 1/00; A61B 17/02; A61B 1/05; A61B 1/307

(52) U.S. Cl. CPC A61B 18/1485; A61B 1/00195; A61L 31/026; A61L 31/06; A61B 2018/00982; A61M 1/008; A61B 17/0218; A61B 1/05; A61B 1/307; A61M 3/0279

(57) ABSTRACT

A resectoscope includes a handle portion, a flow sheath, and an electrode. The flow handle portion includes a trigger, the flow sheath extends from the handle portion and has a hub and a shaft extending from the hub including a rigid inner tube and a semi-rigid outer tube, the rigid inner tube extending through the semi-rigid outer tube to define a channel therebetween, wherein the rigid inner tube may include a fiber-reinforced material, the semi-rigid outer tube may include a polymeric material. The retractor tool is slidably disposed in the flow sheath and includes a linkage and a traction member pivotably coupled to the linkage. The electrode is slidably disposed in the flow sheath and may be made at least in part of sheet metal.

### CROSS REFERENCE TO RELATED APPLICATION

The present application claims the benefit of and priority to U.S. Provisional Application Ser. No. 62/622,231, filed on Jan. 26, 2018, the entire contents of which are incorporated herein by reference.

### BACKGROUND

### 1. Technical Field

The present disclosure generally relates to resectoscopes, and more specifically, to components for use with resectoscopes during surgical procedures.

### 2. Background of Related Art

A resectoscope is typically utilized during the course of a surgical procedure, e.g., a prostate or intrauterine surgical procedure, for removing tissue. Conventional resectoscopes typically include a tubular sheath, one or more electrodes extending through the tubular sheath. A distal end of the tubular sheath is configured to be inserted into a patient to a surgical site. The endoscope includes a lens or an image capturing device at a distal end and an eyepiece at a proximal end and may be inserted into the tubular sheath to extend there through. The electrodes likewise extend through the tubular sheath with one or more of the electrodes including a loop end extending from a wire. One or more handles on an opposite end of the wire from the loop end enable manipulation the loop end relative to the tubular sheath.

During the surgical procedure, a surgeon advances or retracts the resectoscope to a desired position within the surgical site. To aid in suitably positioning the distal end of the tubular sheath, the surgeon can view the surgical site by peering through the endoscope eyepiece. Once the distal end of the tubular sheath is positioned, usually with the aid of a guide placed down the flow sheath, the surgeon may manipulate the one or more handles at the proximal end of the tubular sheath to advance or retract the one or more electrodes relative to target tissue. During advancement and/or retraction, the one or more electrodes are energized to thereby resect the target tissue. In some cases, the surgeon may push or lift tissue with the distal end of the tubular sheath to improve access to the target tissue.

### SUMMARY

Although conventional resectoscopes operate adequately in terms of resecting tissue, they may be improved in aspects of improving removal of tissue through the lumen of the resectoscope sheath, retracting and blunt dissecting tissue. In particular, due to the finite dimensions of the urethra or other naturally-formed opening which may be accessed during a minimally-invasive surgical procedure, an outer diameter of the tubular sheath of the resectoscope is preferably configured to match or be less than the diameter of the opening. As a result, conventional resectoscopes are typically employed for the advancement of an electrode and the housing of an endoscope, with little room for other surgical tools. Accordingly, there is a need for a resectoscope having a configuration capable permitting the advancement of additional tools and improving the out flow of fluid with tissue morsels entrained.

Described herein is a resectoscope, and components thereof, such as a flow sheath, a retractor tool, and an electrode, which addresses the above-mentioned needs and more.

According to embodiments of the present disclosure, a flow sheath for a resectoscope is provided including a hub and a shaft. The shaft extends from the hub and includes a rigid inner tube and a semi-rigid outer tube. The rigid inner tube extends through the semi-rigid outer tube to define a channel therebetween. The rigid inner tube includes a fiber-reinforced material, and the semi-rigid outer tube includes a polymeric material.

In an aspect of the present disclosure, the semi-rigid outer tube may be attached to an outer surface of a distal portion of the rigid inner tube.

According to another aspect of the present disclosure, the semi-rigid outer tube may have a distal portion that extends over a distal end of the rigid inner tube to form a rounded tip.

In another aspect of the present disclosure, the semi-rigid outer tube may include a plurality of apertures formed in a distal portion thereof.

According to another aspect of the present disclosure, the semi-rigid outer tube may be sufficiently sealed to an outer surface of a distal portion of the rigid inner tube to force the irrigation fluid mostly through the plurality of apertures.

In still another aspect of the present disclosure, the hub may include a housing and a curved member, the housing has a main channel and a cavity, the main channel receives proximal portions of semi-rigid outer tube and the rigid inner tube, and the curved member extends radially outwardly from the housing and includes a first channel and a second channel. The rigid inner tube includes a port formed in a side thereof, and the semi-rigid outer tube includes a port formed in a side thereof located proximal of the port of the rigid inner tube. The port of the rigid inner tube disposed is in the cavity of the housing to provide communication between the first channel and a lumen defined by the rigid inner tube. The port of the semi-rigid outer tube is aligned with the second channel.

In yet another aspect of the present disclosure, the first channel and the second channel may be arranged in a side-by-side manner.

In another aspect of the present disclosure, the hub and the shaft may be welded together.

In yet another aspect of the present disclosure, the fiber-reinforced material from which the rigid inner tube is made may comprise carbon-reinforced glass.

In still yet another aspect of the present disclosure, the polymer from which the semi-rigid outer tube is made may include polytetrafluoroethylene.

In another aspect of the present disclosure, an inner surface of the rigid inner tube may be insulative.

According to another embodiment of the present disclosure, a resectoscope is provided including a handle portion and a flow sheath extending from the handle portion. The flow sheath includes a hub, and a shaft extending from the hub including a rigid inner tube and a semi-rigidouter tube. The rigid inner tube extends through the semi-rigid outer tube to define a channel therebetween, the rigid inner tube includes a fiber-reinforced material, and the semi-rigid outer tube includes a polymeric material. A cross-sectional area of the rigid inner tube is from about 70% to about 95% a cross-sectional area of the semi-rigid outer tube.

In another aspect of the present disclosure, the handle portion may include a trigger.

In still another aspect of the present disclosure, the resectoscope may include a retractor tool extending from the trigger of the handle portion.

In another aspect of the present disclosure, the retractor tool may include a linkage coupled to the trigger and a traction member pivotably coupled to the linkage. The traction member includes a plate and a plurality of teeth extending outwardly from the plate at an angle relative to a plane in which the plate lies.

According to still another embodiment of the present disclosure, a retractor tool of a resectoscope is provided including a linkage, and a traction member pivotably coupled to the linkage. The traction member includes a plate and a plurality of teeth extending outwardly from the plate at an angle relative to a plane in which the plate lies.

In another aspect of the present disclosure, each tooth of the plurality of teeth may have a squared-off end and is spaced apart from an adjacent tooth.

In still another aspect of the present disclosure, the retractor tool may further include a trigger, and a wire coupled to and extending between the traction member and the trigger, wherein the trigger is configured to be manipulated in a longitudinal motion to move the traction member longitudinally and to be manipulated in a pull or push motion to pivot the traction member laterally relative to the linkage.

According to still another embodiment of the present disclosure, a resectoscope is provided including a handle portion including a trigger, and a retractor tool extending from the trigger of the handle portion. The retractor tool includes a linkage coupled to the trigger, and a traction member pivotably coupled to the linkage. The traction member includes a plate and a plurality of teeth extending outwardly from the plate at an angle relative to a plane in which the plate lies.

According to still another embodiment of the present disclosure, an electrode of a resectoscope is provided including two longitudinally extending wires, and a wire loop extending from the two longitudinally extending wires. The two longitudinally extending wires and the wire loop are made at least in part from sheet metal.

In an aspect of the present disclosure, the wire loop may be bent at an angle relative to the two longitudinally extending wires to form two corners.

In another aspect of the present disclosure, the two longitudinally extending wires and the wire loop may be coated with a material more conductive than the sheet metal.

According to still another embodiment of the present disclosure, a resectoscope is provided including a handle portion including a trigger, a flow sheath extending from the handle portion, a retractor tool disposed in the flow sheath, and an electrode slidably disposed in the flow sheath. The flow sheath includes a hub, and a shaft extending from the hub including a rigid inner tube and a semi-rigid outer tube, the rigid inner tube extending through the semi-rigid outer tube to define a channel therebetween. The rigid inner tube is made at least in part from a fiber-reinforced material, and the semi-rigid outer tube is made at least in part from a thermoplastic polymer material. A cross-sectional area of the rigid inner tube is about 70% to about 95% of a cross-sectional area of the semi-rigid outer tube. The retractor tool includes a linkage and a traction member pivotably coupled to the linkage. The traction member includes a plate and a plurality of teeth extending outwardly from the plate at an angle relative to a plane in which the plate lies. The electrode is made at least in part from sheet metal.

In an aspect of the present disclosure, the resectoscope may further include an endoscope including an optical guide tube extending through the flow sheath and having an image capture device at a distal end and an eyepiece coupled to the handle portion.

In another aspect of the present disclosure, the retractor tool, the electrode, and the endoscope may be configured to be disposed in the flow sheath at the same time.

In still another aspect of the present disclosure, the retractor tool may be configured to occupy more of a radial cross-sectional area of the lumen of the rigid inner tube than the electrode and the endoscope.

In still yet another aspect of the present disclosure, the retractor tool may be configured to occupy about 50% of the radial cross-sectional area of the lumen of the rigid inner tube.

In still yet another aspect of the present disclosure, the image capture device may be disposed between the two longitudinally extending wires of the electrode.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a resectoscope, in accordance with an exemplary embodiment of the present disclosure;

FIG. 2 is a close-up view of a distal portion of the resectoscope illustrated in FIG. 1;

FIG. 3 is a longitudinal cross section view of the flow sheath of the resectoscope of FIG. 1;

FIG. 4 is a close-up, longitudinal cross section view of a distal portion of the flow sheath, according to an embodiment;

FIG. 5 is a perspective view of a portion of the resectoscope of FIG. 1 with the flow sheath removed;

FIG. 6 is a close-up view of a distal portion of a retraction device and an endoscope, according to an embodiment; and

FIG. 7 is close-up view of the distal portion of the resectoscope illustrated in FIG. 1 disposed in another position.

### DETAILED DESCRIPTION

The present disclosure is directed to a resectoscope for surgically resecting tissue. Specifically, in embodiments the resectoscope includes a flow sheath, an electrode, a retraction device, and an endoscope. The flow sheath is configured to maximize usable space within its outer diameter to enable the incorporation of other surgical instruments/devices, such as the electrode, the retraction device, and the endoscope, within its inner diameter. The electrode may be formed from a cost-effective material that improves resecting of tissue. The retraction device improves the ability of a surgeon to perform bipolar prostatic enucleation of tissue or other surgical procedures, by allowing the surgeon to lift and move tissue for resection. These and other aspects and features of the present disclosure are detailed herein below.

Embodiments of the resectoscope are described in detail with reference to the drawings in which like reference numerals designate identical or corresponding elements in each of the several views. Throughout this description, the term “proximal” refers to the portion of the device or a component thereof that is farther away from the patient and the term “distal” refers to the portion of the device or component thereof that is closer to the patient.

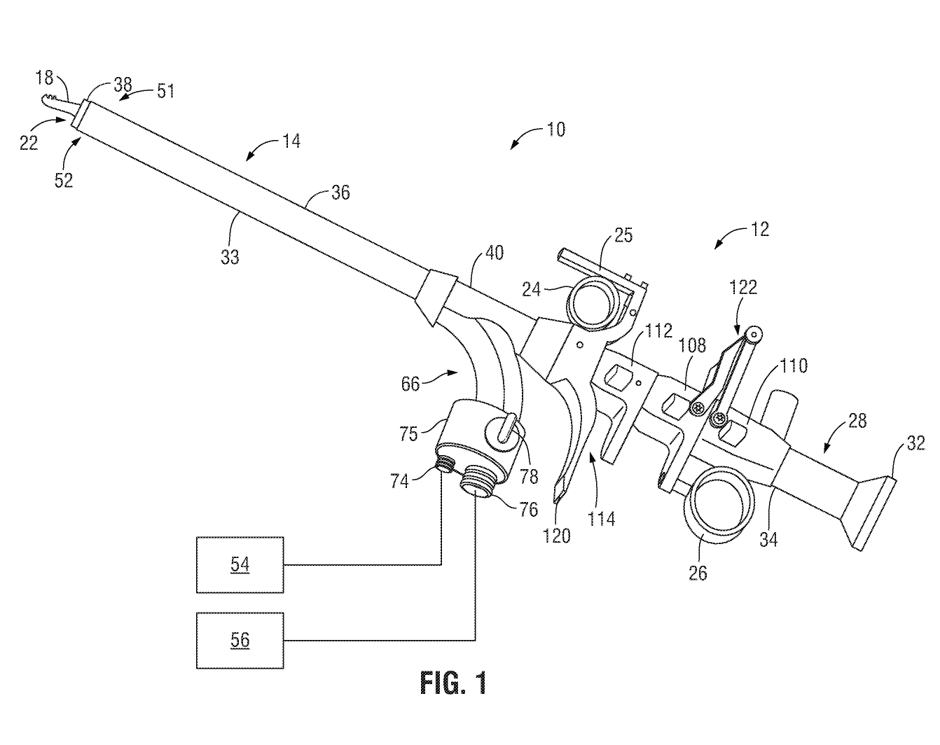

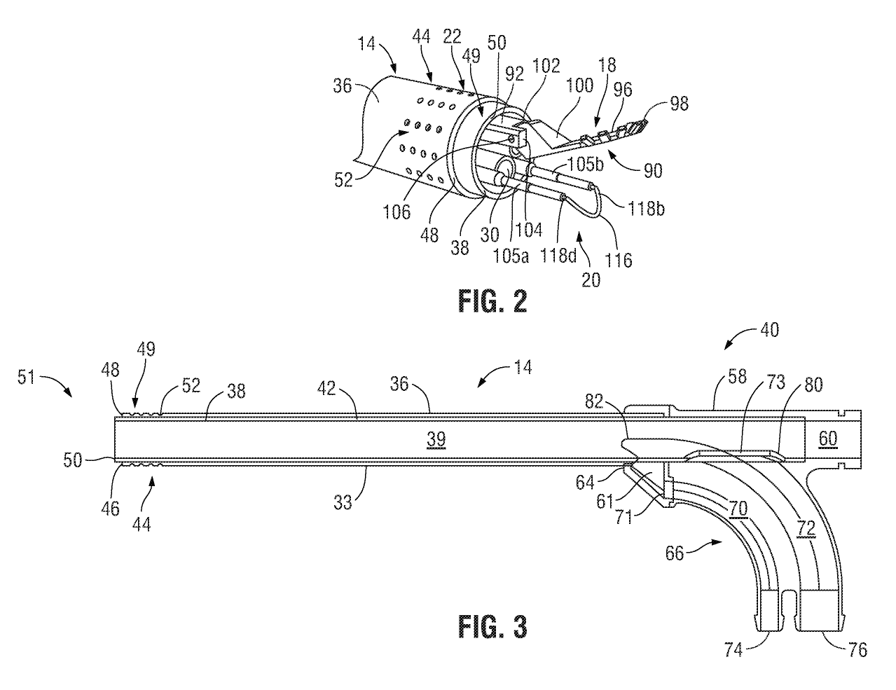

With reference to FIGS. 1 and 2, an exemplary embodiment of a resectoscope 10 is provided for insertion into a patient and advancement to a surgical site to permit surgical tools to be guided to a target. Resectoscope 10 is made up of several components that, when assembled together, form a handle portion 12 and a flow sheath 14 extending distally from handle portion 16. Flow sheath 14 may house surgical tools, such as a retractor tool 18 and an electrode 20, which are each operably coupled to handle portion 12 and slidably disposed within flow sheath 14. Those skilled in the art reading this disclosure will envision other surgical tools that may be housed within flow sheath 14.

Retractor tool 18 and electrode 20 may be advanced out of a distal opening 22 of the flow sheath 14. In this regard, in addition to facilitating the grasping of resectoscope 10, handle portion 12 may facilitate the manipulation of retractor tool 18, via trigger 24, and/or manipulation of electrode 20, e.g., via thumb ring 26 of the handle portion 12, to extend or retract each tool from flow sheath 14. An endoscope 28, which includes an image capture device 30 and an eyepiece 32, may be removably located within flow sheath 14. In particular, image capture device 30 may be disposed at distal opening 22 of flow sheath 14 (as shown in FIG. 2), while eyepiece 32 may be coupled to a proximal end portion 34 of handle portion 12. Thus, the surgeon may peer through eyepiece 32 for a view of the surgical site during a surgical procedure.

Flow sheath 14 is generally configured to provide a defined pathway to guide tools to the target and to provide irrigation and suction to the surgical site, during the surgical procedure. According to an embodiment, flow sheath 14 is made up of a shaft 33 extending from a hub 40. The shaft 33 includes an outer tube 36 and an inner tube 38 extending through outer tube 36 to define an annular channel 42 therebetween. Annular channel 42 is configured to supply irrigation fluid to the surgical site. To maximize the usable space within the outer dimensions of flow sheath 14, outer tube 36 is formed of polytetrafluoroethylene (PTFE) or similar thermoplastic polymer having high strength, toughness, and low friction. Outer tube 36 may be extruded or otherwise extruded and formed and may have a wall thickness in a range of from about 0.05 mm to about 0.5 mm, in an embodiment. In another embodiment, the wall thickness is in a range of, from about 0.1 mm to about 0.25 mm. In still another embodiment, the wall thickness is about 0.2 mm. In this way, outer tube 36 serves as a high strength, thin film over inner tube 38 to permit flow sheath 14 to be easily inserted and removed from the patient.

Inner tube 38 provides a lumen 39 through which tools may be advanced and fluid, tissue, and debris may be aspirated from the surgical site. In this regard, inner tube 38 is configured to provide stiffness to flow sheath 14. Inner tube 38 is formed from a fiber-reinforced material, such as carbon fiber, glass fiber, carbon with a glass insulating surface, and the like. In an embodiment, at least a portion of the inner surface of inner tube 38 is coated with insulating materials. As a result, electrical isolation is provided when electrode 20 is energized. Inner tube 38 has a greater wall thickness than outer tube 36, for example, in a range from about 0.2 mm to about 1.0 mm, in an embodiment. In another embodiment, the wall thickness of the inner tube 38 is in a range of from about 0.3 mm to about 0.5 mm. In still another embodiment, the wall thickness of the inner tube 38 is about 0.44 mm. The inner tube 38 has a radial cross-sectional area that may be from about 10% to about 90%, about 70% to about 95% or from about 70% to about 80% of a radial cross-sectional area of outer tube 36, in an embodiment. In another embodiment, when utilized with an instrument having a size of 24 french, the inner tube 38 has a radial cross-sectional area that may be from about 85% to about 95% of the radial cross-sectional area of outer tube 36. As can be appreciated, the radial cross-sectional area of the inner tube 38 may vary according to the size of the instrument being utilized by the clinician.

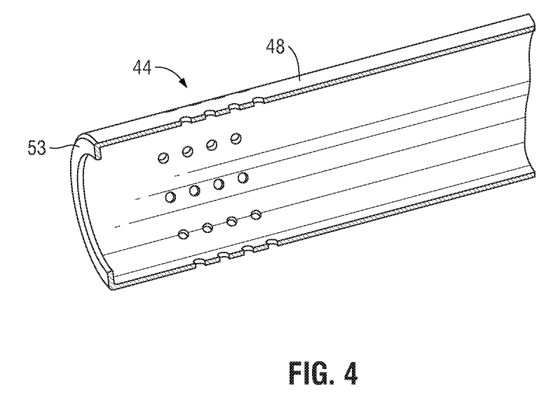

Outer and inner tubes 36, 38 may extend coaxially, in an embodiment. In another embodiment, outer and inner tubes 36, 38 are co-extensive, but offset relative to each other. Outer tube 36 has a distal portion 44 with a distal opening 46 formed at distal end 48 of outer tube 36. Outer tube 36 and inner tube 38 may be coupled to each other at their respective distal portions 44, 49. In an embodiment, distal end 48 of outer tube 36 is coupled to an outer surface of distal portion 49 of inner tube 38 such that outer tube 36 ends just short of distal end 50 of inner tube 38. For example, distal end 48 of outer tube 36 may be located from about 0.1 mm to about 10 mm, in an embodiment, or from about 1 mm to about 5 mm, in another embodiment, from distal end 50 of inner tube 38, and outer tube 36 may be welded, for example, ultrasonically welded, or adhered, or otherwise coupled to inner tube 38. As a result, a distal portion 51 of flow sheath 14 forms a blunt tip. In another embodiment, as shown in FIG. 4, distal portion 44 of outer tube 36 is folded over to cover distal end of 50 of inner tube 38 and to provide flow sheath 14 with an edge 53 that wraps around distal end 50 of inner tube 38. Edge 53 is configured to soften the tip of flow sheath 14 which may reduce irritation of the urethra as the resectoscope is being inserted therein. In an embodiment, edge 53 may be secured to the inner surface of inner tube 38 forming a rounded tip for flow sheath 14.

As illustrated in FIGS. 1 and 2, irrigation apertures 52 are formed in distal portion 44 of outer tube 36. Irrigation apertures 52 may be formed around an entire circumference of outer tube 36 as four rings, in an embodiment. Alternatively, irrigation apertures 52 may form more or fewer rings and/or form a different pattern on distal portion 48 of outer tube 36. In any case, irrigation apertures 52 are configured to expel irrigation fluid fed through channel 42 to the surgical site during the surgical procedure.

Hub 40 couples channel 42 to irrigation fluid source 54 and lumen 39 of inner tube 38 to an outflow controller 56, respectively. As such, hub 40 is made up of a housing 58 having a main channel 60 for receiving proximal portions of outer and inner tubes 36, 38 in a cavity 61. Housing 58 includes a distal opening 64 which permit surgical tools to advance through main channel 60 and allows endoscope 28 to extend therethrough. A curved member 66 extends radially outwardly from housing 58 and has two separate channels, namely, an irrigation channel 70 and an outflow channel 72, formed therein. Irrigation and outflow channels 70, 72 are arranged side-by-side for a more compact design as compared to resectoscopes having irrigation and outflow channels 70, 72 that are arranged opposite from or perpendicular to each other. Each of irrigation and outflow channels 70, 72 has an outlet 71, 73, respectively, which may be disposed 90° relative to each other. In an embodiment, as part of the compact design of hub 40, outlet 71 is arranged substantially parallel to flow sheath 14, and outlet 73 is arranged substantially perpendicular to flow sheath 14. In another embodiment, outlets 71, 73 are at another position relative to one another. In an embodiment, a cross-sectional area of irrigation channel 70 is greater than a cross-sectional area of outflow channel 72. In another embodiment, the cross-section areas of each channel 70, 72 are substantially equal to each other.

The outflow controller 56 enables fluid flowing through the irrigation channel 70 to enter the surgical site and expand the surgical site (e.g., the urethra, bladder, uterus, etc.) by obstructing the outflow channel 72. The obstruction may be under the direct control of the surgeon via a valve (not shown). In embodiments, the outflow controller 56 may have a upstream pressure control regulator (not shown) to maintain constant pressure within the surgical site, or a flow meter to maintain input and output flow through the irrigation channel 70 and the outflow channel 72, respectively. It is contemplated that the outflow of fluid may be regulated in a pulsatile manner such that the fluid is allowed to discharge at maximum flow rate to enable debris from the resection process to be cleared before obstructing the outflow channel 72 to enable the surgical site to recharge with clean fluid. As can be appreciated, an integrated control loop (not shown) may increase the efficacy of the pulsatile flow of fluid such that the outflow of fluid from the surgical site matches the inflow of fluid to the surgical site. It is contemplated that one or more pressure sensors (not shown) may be disposed in proximity to the surgical site to enable more accurate matching of the inflow and outflow of fluid to and from the surgical site. In embodiments, a net inflow and outflow may be measured by the loss of volume or weight from the irrigation fluid source 54 and fluid captured from the outflow channel 72 downstream of the outflow controller 56. In one non-limiting embodiment, the outflow controller 56 includes a vacuum source to draw fluid through the outflow channel 72 from the surgical site.

A connector piece 75 is coupled to or integrally formed on curved member 66 and includes two connector ports 74, 76. Irrigation channel 70 provides communication between connector port 74 and cavity 61, while outflow channel 72 provides communication between connector port 76 and main channel 60. A dial or knob 78 is mounted to an outer surface of connector piece 75 to permit a user to control a valve (not shown) disposed in outflow channel 72 for regulating the amount of suction provided.

With continued reference to FIG. 3, outer tube 36 includes a suction port 80, and inner tube 38 includes an irrigation port 82 proximal to suction port 80, each port 80, 82 being formed on a respective side of outer tube 36 and inner tube 38. Thus, when tubes 36, 38 are positioned within hub 40, irrigation port 82 is disposed within cavity 61 to permit fluid source 54 to provide irrigation fluid to channel 42 via outlet 71, while suction port 80 aligns with outlet 73 of outflow channel 72 to permit outflow controller 56 to provide suction to lumen 39 of inner tube 38.

Hub 40 and flow sheath 14 may be formed as two separate components and coupled together, for example, by ultrasonic weld and the like, in an embodiment. In another embodiment, portions of hub 40 and flow sheath 14 are integrally formed, and other parts, for example, connector piece 75, are coupled to hub 40 and flow sheath 14.

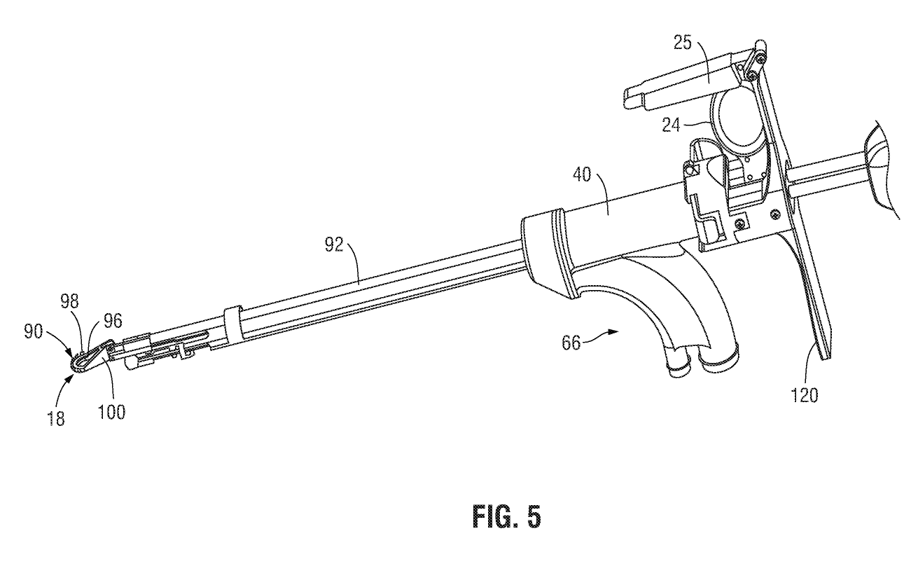

By maximizing the cross-sectional area of inner tube 38 while maintaining the outer diameter of outer tube 36 the same as or less than prior art flow sheaths, a greater number of surgical tools may be housed within and thus, introduced, into a surgical site during a surgical procedure. For example, as briefly noted above, retractor tool 18 is housed in flow sheath 14. With reference to FIGS. 1, 2, 5, and 6, retractor tool 18 is configured to permit the surgeon to not only push tissue that may be positioned in front of flow sheath 14, but also lift tissue in a desired manner. In this regard, retractor tool 18 includes a traction member 90 pivotably coupled to a linkage 92 extending through flow sheath 14 to a trigger 24 disposed in handle portion 12.

To facilitate traction, traction member 90 includes a plate 96 having teeth 98 extending outwardly at an angle relative to the plane in which the plate 96 lies. For example, teeth 98 may extend outwardly at an angle from about 10 to about 90 degrees, in an embodiment relative to the plane of plate 96. In another embodiment, teeth 98 extend outwardly at an angle from about 45 to about 60 degrees relative to the plane of plate 96. Each tooth 98 has a squared-off end portion and is spaced apart from an adjacent tooth to thereby provide a jagged but atraumatic surface for catching and retracting tissue, in an embodiment. In another embodiment, each tooth 98 has a different shape suitable to provide a rough surface to more easily catch and retract tissue during a surgical procedure.

To attach plate 96 to linkage 92 and hence, to trigger 24, traction member 90 has an arm portion 100 extending proximally from plate 96 into a notch 102 at a distal end portion 104 of linkage 92. A pivot pin 106 is inserted through openings of distal end portion 104 of linkage 92 and through corresponding openings on arm portion 100 to pivotably couple traction member 90 to linkage 92. According to an embodiment, to permit the movement of plate 96 relative to linkage 92, arm portion 100 is also coupled to a cable, rod, wire or other connection-type of mechanism (not shown) that is inserted through hole 103 at a position offset relative to pivot pin 106. The connection-type mechanism may extend through or alongside linkage 92 or may be disposed in another co-extensively aligned tube. No matter the particular configuration, the connection-type mechanism extends an entire length of flow sheath 14 through hub 40 to couple plate 96 to trigger 24.

Trigger 24 is movably coupled to a rotatable cylinder 114 rotatably coupled to hub 40. In addition to trigger 24, a grip 120 may extend radially from rotatable cylinder 114 to permit the surgeon to more easily grasp resectoscope 10. Trigger 24 may be spring-loaded to bias traction member 90 to a resting position. The resting position may be one in which traction member 90 defines a minimum angle relative to a longitudinal axis of linkage 92, in an embodiment. For example, according to an embodiment, traction member 90 may have a resting position during which plate 96 is positioned at about a 10° angle relative to linkage 92 (see, e.g., FIG. 7), and actuation of trigger 24 may pivot plate 96 to about 30° relative to linkage 92 (see, e.g., FIG. 2). Alternatively, the resting position may be one in which traction member 90 is positioned at a maximum angle so that traction member 90 is disposed at a maximum-allowed angle relative to the plane of linkage 92. In any case, during operation, the surgeon may manipulate trigger 24, for example, by moving trigger 24 in a lateral motion relative to handle portion 12 to cause traction member 90 to pivot relative to linkage 92 in a corresponding direction. Further, a ratchet mechanism 25 may be provided to enable incremental locking of trigger 24 relative to rotatable cylinder 114, thus enabling locking of plate 94 in various incremental angled positions.

Traction member 90 and arm portion 100 are made of stainless steel or another engineering resin such as a thermoplastic polymer, including polyether ether ketone (PEEK), or Liquid Crystal Polymer (LCP) biocompatible materials. In accordance with an embodiment, traction member 90 and arm portion 100 are formed of the same material.

As shown in FIG. 2, traction member 90 is configured to occupy more of the radial cross-sectional area than endoscope 28 and electrode 20. For example, traction member 90 is configured to occupy about half (e.g., about 50%) of the radial cross-sectional area of lumen 39, while endoscope 28 (for example, image capture device 30) and electrode 20 reside in the remaining half of lumen 39. In an embodiment, image capture device 30 is disposed between two wires 105a, 105b of electrode 20 and between electrode 20 and traction member 90, and movement of traction member 90 is confined to directions towards and from image capture device 30, generally perpendicular relative to a plane defined through wires 105a, 105b. Thus, in contrast to prior art resectoscope configurations, three tools may be disposed in flow sheath 14 and used by the surgeon at the same time. Moreover, arranging traction member 90, image capture device 30 and electrode 20 in the manner described above permits image capture device 30, and hence the surgeon, with an unobstructed view of the surgical site during a surgical procedure.

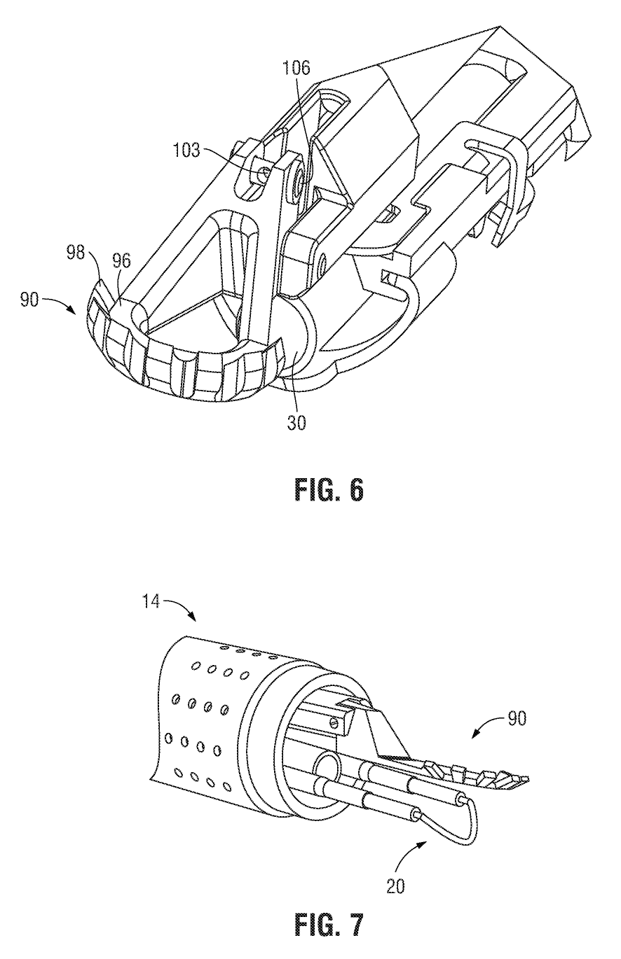

Electrode 20 includes wires that, in embodiments, may be manufactured from sheet metal made of copper, copper alloy, or another material having suitable thermal conductivity. Wires 105a, 105b of electrode 20 include a longitudinal portion extending through inner tube 38 and a wire loop 116 connecting the longitudinal portions of wires 105a, 105b. Wire loop 116 is bent relative to the longitudinal portion to form two corners 118a, 118b. In accordance with an embodiment, wire loop 116 is perpendicular relative to wires 105a, 105b. In another embodiment, wire loop 116 is bent at a different angle, for example, between about 30 degrees and about 60 degrees, relative to wires 105a, 105b. Wires 105a, 105b and/or wire loop 116 of electrode 20 may be coated with a highly conductive material (for example, a material more conductive than sheet metal) or an epoxy coating, which in conjunction with the inclusion of the jutting out corners 118a, 118b improves the concentration of an electric field around wire loop 116 to provide a coagulative effect during the surgical procedure. Specifically, in an embodiment such as depicted in FIG. 6 electrode 20 is formed from sheet metal and is coated in an insulating material such as a fluoropolymer or high temperature epoxy such that only the cutting edge protrudes from the coating. This effect is enhanced by tapering the thermally conductive sheet metal, typically of copper or niobium alloy to a thin edge to intensify the electrosurgical effect. An included angle on the cutting edge of approximately 20° may be employed. Concave faces leading to the angle help with heat sinking structural integrity of the edge.

To operate electrode 20, the surgeon manipulates thumb ring 26 secured to a guide plate 108, which is coupled to a proximal end of electrode 20. In an embodiment, guide plate 108 is slidable along endoscope 28, which includes eyepiece 32, extends through flow sheath 14, and includes image capture device 30 at its distal end. In embodiments, endoscope 28 also includes an eyepiece connector 110 and extends through a sliding body 112 to which a proximal end portion of electrode 20 is coupled, rotatable cylinder 114, hub 40, and inner tube 38. A pivoting bridge 122 couples sliding body 112 to eyepiece connector 110 and is configured to expand and collapse, in response to actuation of thumb ring 26. As such, when thumb ring 106 is advanced distally, bridge 122 expands to thereby cause electrode 20 to retract relative to inner tube 38, and when thumb ring 26 is moved in an opposite direction along endoscope 28, bridge 122 collapses and causes electrode 20 to advance relative to inner tube 38.

During a surgical procedure, such as a transurethral resection of the prostate (“TURP”), resectoscope 10, in particular, flow sheath 14 is inserted into a patient's urethra until distal opening 22 of flow sheath 14 is positioned adjacent target tissue. Outflow control for suction and fluid for irrigation may be supplied to the surgical site through flow sheath 14, in an embodiment. The fluid, for example, saline, flowing in channel 42 located between outer tube 36 (for example, PTFE layer) and inner tube 38 (for example, carbon fiber tube) flushes the surgical site so that tissue, such as blood, does not interfere with the surgeon's view. The fluid also creates a fluid pocket around the distal end of the resectoscope 10 to thereby facilitate surgical navigation. The fluid is removed from the surgical site through lumen 39 by suction. Meanwhile, flow sheath 14 remains stationary in the patient.

Due to the increased inner diameter of inner tube 38 and decreased wall thicknesses of both inner and outer tubes 38, 36, surgical tools such as electrode 20 and traction member 90 may be inserted into flow sheath 14 alongside endoscope 30 and rotated freely within. As such, retractor tool 18, electrode 20, and endoscope 28 are configured to be disposed in the flow sheath 14 at the same time. In addition to applying forward pressure from flow sheath 14 to push tissue, traction member 90 provides traction across the line of dissection (for example, the prostate capsule during TURP). Traction member 90 provides traction so that tissue is lifted away from the intended plane of dissection, which may be opened as electrode 20 is advanced. Further, a bulk of the prostate tissue is held out of view of image capture device 30 so the surgeon is provided with a clear view of the surgical site. Thus, relatively large portions of tissue may be removed during the procedure, compared to the relatively thin shaved pieces of the prostate tissue that may be removed using previous resectoscopes. Additionally, the smooth flow path and increased space for the exiting saline allows the user to remove pieces of resected tissue during the procedure.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto

What is claimed is:

1. A flow sheath of a resectoscope, comprising:
a hub; and
a shaft extending from the hub and including a rigid inner tube and a semi-rigid outer tube, the ridged inner tube extending through the semi-rigid outer tube to define a channel therebetween,
the rigid inner tube including a fiber-reinforced material, and the semi-rigid outer tube including a polymeric material.

2. The flow sheath of claim 1, wherein the semi-rigid outer tube has a distal portion that extends over a distal end of the rigid inner tube to form a rounded tip.

3. The flow sheath of claim 1, wherein the semi-rigid outer tube includes a plurality of apertures formed in a distal portion thereof.

4. The flow sheath of claim 3, wherein the semi-rigid outer tube is sufficiently sealed to an outer surface of a distal portion of the rigid inner tube to force the irrigation fluid mostly through the plurality of apertures.

5. The flow sheath of claim 1, wherein:
the hub includes a housing and a curved member, the housing has a main channel and a cavity, the main channel receives proximal portions of the semi rigid outer tube and the rigid inner tube, and the curved member extends radially outwardly from the housing and includes a first channel and a second channel,
the rigid inner tube includes a port formed in a side thereof, the semi-rigid outer tube includes a port formed in a side thereof located proximal of the port of the rigid inner tube,
the port of the rigid inner tube disposed in the cavity of the housing to provide communication between the first channel and a lumen defined by the rigid inner tube, and
the port of the semi-rigid outer tube is aligned with the second channel.

6. The flow sheath of claim 5, wherein the first channel and the second channel are arranged in a side-by-side manner.

7. The flow sheath of claim 1, wherein the hub and the shaft are welded together.

8. The flow sheath of claim 1, wherein the fiber-reinforced material comprises carbon-reinforced glass.

9. The flow sheath of claim 1, wherein the polymer comprises polytetrafluoroethylene.

10. The flow sheath of claim 1, wherein an inner surface of the rigid inner tube is insulative.

11. A resectoscope, comprising:
a handle portion; and
a flow sheath extending from the handle portion, the flow sheath comprising:
a hub, and
a shaft extending from the hub and including a rigid inner tube and a semi-rigid outer tube, the rigid inner tube extending through the semi-rigid outer tube to define a channel therebetween, the rigid inner tube including a fiber-reinforced material, and the semi-rigid outer tube including a polymeric material.

12. The resectoscope of claim 11, wherein the handle portion includes a trigger.

13. The resectoscope of claim 12, further including a retractor tool extending from the trigger of the handle portion.

14. The resectoscope of claim 13, wherein the retractor tool includes a linkage coupled to the trigger and a traction member pivotably coupled to the linkage, the traction member including a plate and a plurality of teeth extending outwardly from the plate at an angle relative to a plane in which the plate lies.

15. A resectoscope, comprising:
a handle portion including a trigger;
a flow sheath extending from the handle portion, wherein the flow sheath includes a hub, and a shaft extending from the hub and including a rigid inner tube and a semi-rigid outer tube, the rigid inner tube extending through the semi-rigid outer tube to define a channel therebetween, the rigid inner tube made at least in part of a fiber-reinforced material, and the semi-rigid outer tube made at least in part of a polymeric material;
a retractor tool disposed in the flow sheath including a linkage and a traction member pivotably coupled to the linkage, the traction member including a plate and a plurality of teeth extending outwardly from the plate at an angle relative to a plane in which the plate lies, and
an electrode slidably disposed in the flow sheath, the electrode comprising sheet metal.

16. The resectoscope of claim 15, further comprising an endoscope including an optical guide tube extending through the flow sheath and having an image capture device at a distal end and an eyepiece coupled to the handle portion.

17. The resectoscope of claim 16, wherein the retractor tool, the electrode, and the endoscope are configured to be disposed in the flow sheath at the same time.

18. The resectoscope of claim 17, wherein the retractor tool is configured to occupy more of a radial cross-sectional area of the lumen of the rigid inner tube than the electrode and the endoscope.

19. The resectoscope of claim 18, wherein the retractor tool is configured to occupy about 50% of the radial cross-sectional area of the lumen of the rigid inner tube.

20. The resectoscope of claim 16, wherein the image capture device is disposed between the two longitudinally extending wires of the electrode.
